(19) United States
(12) Patent Application Publication
(10) Pub. No.: US 2019/0318290 A1
(43) Pub. Date: Oct. 17, 2019

(54) MEDICAL TRANSFER PROTOCOL SYSTEM AND METHOD

(71) Applicants: Jeffrey Clawson, Holladay, UT (US); Ivan Whitaker, Woods Cross, UT (US); Brett Patterson, Clearwater, FL (US); Mark Rector, Murray, UT (US); Darren Judd, Coalville, UT (US)

(72) Inventors: Jeffrey Clawson, Holladay, UT (US); Ivan Whitaker, Woods Cross, UT (US); Brett Patterson, Clearwater, FL (US); Mark Rector, Murray, UT (US); Darren Judd, Coalville, UT (US)

(21) Appl. No.: 15/953,297

(22) Filed: Apr. 13, 2018

(51) Int. Cl. G06Q 10/06; H04M 3/51; G16H 40/20; G06Q 30/04; G16H 20/10; G16H 40/40

(52) U.S. Cl. CPC G06Q 10/063112; H04M 3/5133; H04M 3/5183; G16H 40/20; H04M 3/5175; H04M 3/5116; G16H 20/10; G16H 40/40; G06Q 30/04

(57) ABSTRACT

A system and method assists a dispatcher in responding to callers requesting medical transfers for a patient. A computer implemented medical transfer protocol includes interrogatories for a dispatcher to ask a caller to generate an appropriate medical transfer. The medical transfer protocol selects a medical vehicle with the appropriate equipment, medicinal inventory, and trained medical personnel.

### COPYRIGHT NOTICE

© 2018 Priority Dispatch Corp. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### TECHNICAL FIELD

The present disclosure relates to computer systems and methods for providing medical transfer interrogation, instruction, and dispatch. More specifically, the disclosure is directed to computer-implemented protocols to enable a dispatcher to process medical transfer requests in an accurate, consistent, and systematic manner by guiding the dispatcher during interrogation, response determination, and caller instruction.

### BRIEF DESCRIPTION OF THE DRAWINGS

FIG. 1 is a block diagram of a medical transfer system, according to one embodiment.

FIG. 2 is a flow diagram of a medical transfer protocol of a medical transfer system, according to one embodiment.

FIGS. 3A-3N illustrate embodiments of a user interface for a medial transfer protocol for an unscheduled medical transfer.

FIGS. 4A-4J illustrate embodiments of a user interface for a medical transfer protocol for a scheduled medical transfer.

FIGS. 5A-5J illustrate embodiment of a user interface for a medical transfer protocol for a mental health transfer.

### DETAILED DESCRIPTION

Patient transfer services are a critical part of medical care when evaluation and care services at higher level or specialized care facilities are needed. For example, a patient in a rest home or long term care facility may require urgent or specialized medical treatment. When a patient is in transit, the patient may need access to medication, equipment, and trained personnel that were not available at a previous residence or facility.

Conventional medical transfer services do not recommend field response apparatuses based on vehicle types, acuity of care, medical equipment, or medications. As such, a vehicle may arrive that is not equipped to accommodate the patient or a vehicle may arrive with more equipment than is needed. For example, a vehicle may include basic life support (BLS) but advanced life support (ALS) is needed for the patient evaluation, care, and the transfer. Alternatively, an emergency medical technician may arrive in the assigned vehicle but a paramedic is needed. As can be appreciated, many different combinations of vehicles, medical personnel, equipment, and medicines are available, and suitable matches are needed to serve each patient and maximize personnel and vehicle response efficiency. Conventional systems require call centers to manually select resources based on available information. Without automated tracking and inventory matching (crew types, vehicles, medications, and equipment), insufficient or excessive resources may be inappropriately assigned to the medical transfer.

In scheduling a medical transfer, a dispatcher conducts an interrogation process with a caller calling on behalf of the patient. As disclosed herein, a medical transfer protocol provides a logic tree with preprogrammed inquiries to remove subjective variations based on individual dispatchers. Accordingly, uniform and consistent results are achieved with routine dispatcher training and protocol use and therefore extensive medical training for these dispatchers is not needed.

As disclosed herein, a medical transfer protocol also provides uniform and consistent billing codes for accurate payment processing and correct insurance reimbursement. The medical transfer protocol automatically selects the appropriate medical crew and vehicle and simultaneously provides a reliable determinant code that corresponds to a specific billing code. The billing code is based on the required equipment, medication, acuity level, and nature of the transport. As with the selection of the vehicle, the billing code is not left to the discretion of a dispatcher. In this manner, the billing code is determined based on the protocol and the determined facts at the time the medical transfer is requested.

The embodiments of the disclosure will be best understood by reference to the drawings, wherein like parts are designated by like numerals throughout. It will be readily understood that the components of the disclosed embodiments, as generally described and illustrated in the figures herein, could be arranged and designed in a wide variety of different configurations. Thus, the following detailed description of the embodiments of the systems and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified.

In some cases, well-known features, structures or operations are not shown or described in detail. Furthermore, the described features, structures, or operations may be combined in any suitable manner in one or more embodiments. It will also be readily understood that the components of the embodiments as generally described and illustrated in the figures herein could be arranged and designed in a wide variety of different configurations.

Several aspects of the embodiments described will be illustrated as software modules or components. As used herein, a software module or component may include any type of computer instruction or computer executable code located within a memory device and/or transmitted as electronic signals over a system bus or wired or wireless network. A software module may, for instance, comprise one or more physical or logical blocks of computer instructions, which may be organized as a routine, program, object, component, data structure, etc., that performs one or more tasks or implements particular abstract data types.

In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. In addition, data being tied or rendered together in a database record may be resident in the same memory device, or across several memory devices, and may be linked together in fields of a record in a database across a network.

Suitable software to assist in implementing the invention is readily provided by those of skill in the pertinent art(s) using the teachings presented here and programming languages and tools, such as Java, Pascal, C++, C, database languages, APIs, SDKs, assembly, firmware, microcode, and/or other languages and tools. Suitable signal formats may be embodied in analog or digital form, with or without error detection and/or correction bits, packet headers, network addresses in a specific format, and/or other supporting data readily provided by those of skill in the pertinent art(s). [0013] A medical dispatch system disclosed herein may be computer-implemented in whole or in part on a digital computer. The digital computer includes a processor performing the required computations. The computer further includes a memory in electronic communication with the processor for storing a computer operating system. The computer operating systems may include MS-DOS, Windows, Unix, AIX, CLIX, QNX, OS/2, and Apple. Alternatively, it is expected that future embodiments will be adapted to execute on other future operating systems. The memory also stores application programs including a Computer Aided Dispatch (CAD) program, an emergency medical dispatch protocol, and a user interface program, and data storage. The computer further includes an output device, such as a display unit, for viewing the displayed instructions and inquiries and as a user input device for inputting response data.

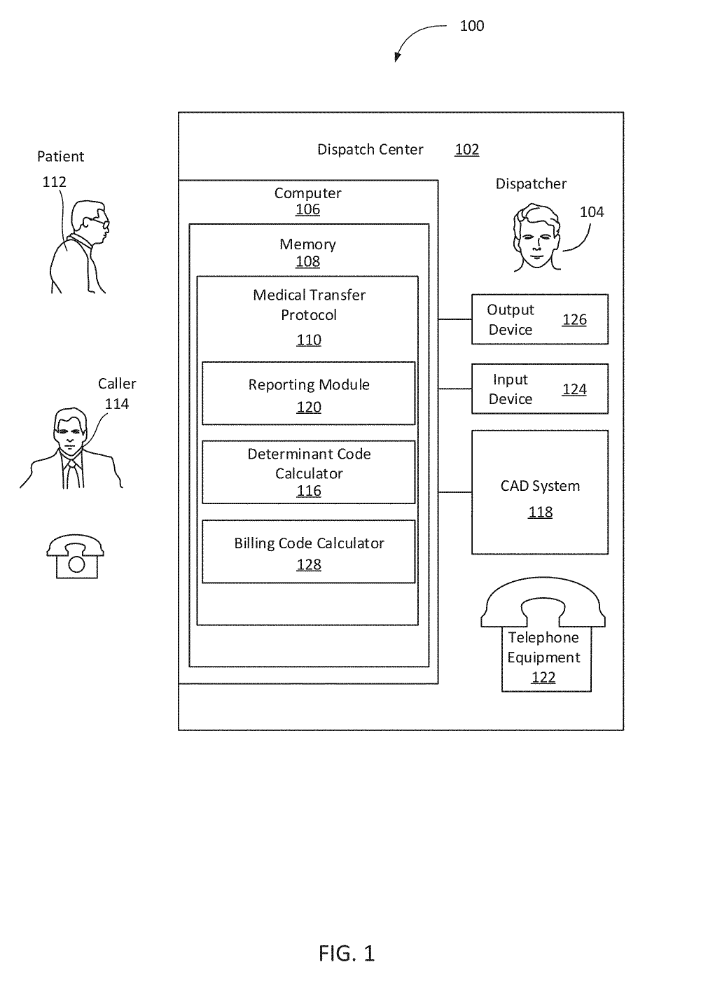

Referring to FIG. 1, one embodiment of a computer-aided medical transfer system 100 is shown. At a dispatcher center 102, a dispatcher 104 operates a computer 106 having a memory 108 with a medical transfer protocol 110 at least partially stored thereon to enable the dispatcher 104 to rapidly and consistently initiate a medical transfer. The medical transfer is a service to deliver a patient 112 from a resident, long term care facility, medical facility and the like to a medical facility for a wide variety of evaluations and treatments. The medical transfer utilizes medical personnel with appropriate training and a service vehicle with support equipment and medicines on board. Unlike conventional systems, the patient 112 is matched with a suitably equipped vehicle and appropriately trained medical personnel. The medical transfer may be due to a sudden urgent need such as a medical emergency or may be scheduled in advance for non-urgent medical treatment, physical check-up, mental health evaluation and counseling, and the like.

The medical transfer protocol 110 is initiated when a dispatcher 104 receives a call from a caller 114 requesting a medical transfer on behalf of the patient 112. In some instances, the patient 112 may call on his or her own behalf. The medical transfer protocol provides a logic tree with questions, possible responses from the caller 114, and possible instructions to the caller 114. The caller responses may in the logic lead to subsequent questions and/or instructions to the caller 114. The responses are processed according to predetermined logic to provide an appropriate medical transfer response. During the medical transfer protocol 110, the dispatcher 104 and/or the medical transfer protocol 110 will gather, inter alia, conditions and circumstances of the medical transfer and the patient's condition, discovered through interrogation, in order to dispatch an appropriate medial transfer service. The medial transfer protocol 110 facilitates uniform and consistent gathering of information relating to the transfer and dispatching of an appropriate medical transfer service. The appropriate medical transfer service may be determined, in part, through a system of logically assigning determinant codes as the protocol progresses (i.e., traverses) through the logic tree.

Exemplary embodiments of medical dispatch protocols with logic trees are disclosed in U.S. Pat. Nos. 5,857,966, 5,989,187, 6,004,266, 6,010,451, 6,053,864, 6,076,065, 6,078,894, 6,106,459, 6,607,481, 7,106,835, 7,645,234, 8,066,638, 8,103,523, 8,294,570, 8,335,298, 8,355,483, 8,396,191, 8,488,748, 8,670,526, 8,712,020, 8,873,719, 8,971,501, 9,319,859, 9,491,605, and 9,516,166 which are incorporated herein by reference.

The medical transfer protocol 110 includes and operates a determinant code calculator 116 to calculate a determinant code from the caller's responses to protocol questions. After processing the call, the determinant code calculator 116 generates a determinant code that indicates the urgency of a medical transfer. The protocol questions and the medical transfer deal directly with life-and-death decisions and the protocols discussed herein pass a rigorous medical review by a panel of doctors and EMS public safety experts who specialize in medical dispatch. The determinant codes may range, for example, from DELTA for generally very serious emergencies to ALPHA for generally less serious emergencies. When a determinant value is identified in one of the four levels (ALPHA—A, BRAVO—B, CHARLIE—C, and DELTA—D) the response configuration (e.g., the medical vehicles involved and the mode of response) is dispatched as indicated by the medical transfer protocol. If the protocol 110 determines that the medical transfer is not urgent, a request is sent to a non-emergency provider instead of dispatching an emergency response vehicle.

As many calls for medical transfer are not medical emergencies, medical transfers are prioritized according to need and available resources. Medical transfers that are emergencies receive a higher priority and merit immediate evaluation and medical transfer. Medical transfers for patients with unique needs for emergency medical evaluation, medicines, medical equipment, vehicle equipment, and suitably trained personnel must be matched with appropriately. If the medical transfer is not urgent then lights-and-siren are not needed and will not be used thereby increasing the safety of all those on the road and in the emergency vehicles. While many medical transfers are not emergencies, all transfers can benefit from evaluation and the appropriate provision of post-dispatch or pre-arrival instructions. In some embodiments, prior to the arrival of the medical transfer, the medical transfer protocol 110 may provide instructions that are appropriate to the type of transfer such as the emergency nature of the transfer, physical condition of the patient, mental condition of the patient, medicinal patient needs, medical equipment needs for the patient, physical assistance needs for the patient, and the like.

The determinant code provides a categorization code of the type and level of the incident, the code is provided to a Computer Aided Dispatch (CAD) system 118, which is a tool used by dispatchers to track and allocate emergency response resources, for processing. The CAD system 112 may manage dispatcher tools for processing emergency calls, including but not limited to the emergency dispatch protocol 110 or the expedited dispatch protocol 116, communication resources (e.g.., radio system, alpha pager), mapping tools (e.g., global positioning system (GPS) technology, geographic information systems (GIS)), and vehicle location systems (e.g., automatic vehicle location (AVL)). The CAD system 118 may operate in whole or in part on a separate computer in communication with computer 106. The primary information used in this task is location information of both the incident and units, unit availability, and the type of incident. CAD systems may use third-party solutions, such as E-911, vehicle location transponders, and MDTs for automating the location and availability tasks.

The computer 106 may include a reporting module 120 to statistically measure the performance of individual staff and overall center performance. The statistics include compliance rates, call processing statistics, and peer measurements.

The dispatch center 102 includes telephony equipment 122, an input device 124, and an output device 126 to respond to calls and interface with the computer 106. The dispatcher 104 receives calls on the telephony equipment 122, identifies a call as requiring a medical transfer, and initiates the medical transfer protocol 110. In scheduling a medical transfer, the dispatcher 104 asks a series of questions and while some questions are intuitive some protocol questions may be missed if the dispatcher 104 is not guided. The medical transfer protocol 110 provides instructions that are expertly drafted to assist a novice caller in determining patient needs and condition to thereby provide a suitable medical transfer. The medical transfer protocol 110 may also provide expertly drafted first aid instructions to assist a caller 114 prior to the arrival of emergency responders.

The medical transfer protocol 110 may further include a billing code calculator 128 to generate a billing code based on the medical transfer service. The billing code calculator 128 receives information regarding the nature of the medical transfer, safety information, the vehicle, including vehicle equipment and medical supplies, the medical personnel, the time and date, the distance, and the nature of the destination. These factors determine a billing code that is generated at the time of request and may be used for invoice generation and insurance submission.

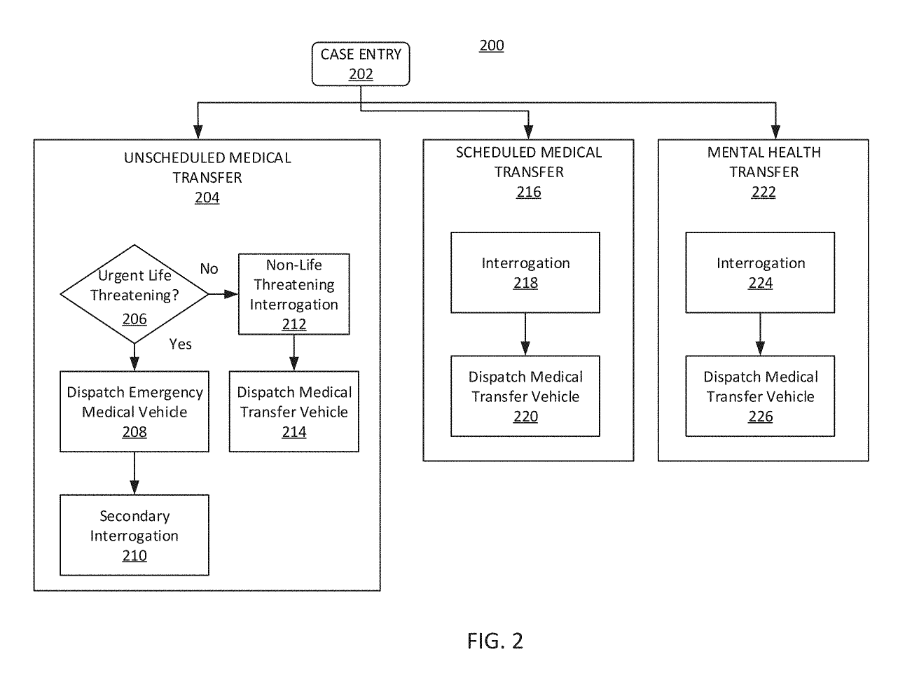

FIG. 2 is a flow diagram 200 of an embodiment of a medical transfer protocol 110. The protocol 110 may begin with Case Entry 202 that guides the dispatcher 104 in gathering initial information. One aim of Case Entry 202 is to obtain sufficient information from the caller 114 to permit identification of the reason for the medical transfer. Also, Case Entry 202 may be considered a primary interrogation because all calls may be processed through the case entry 202 to gather initial patient and transfer information. The information received through the case entry 202 may include the location for pick up, patient contact telephone number, nature of the medical transfer, patient age, patient gender, and any medical issues or complaints. In one embodiment, the Case Entry 202 may include what is referred to in the field of art as “the four commandments of emergency medical dispatching,” the patient's age, the status of the patient's breathing, the status of the patient's consciousness, and a description of the patient's medical condition or chief complaint, if any.

If Case Entry 202 determines that the medical transfer is due to an acute medical problem, the protocol 200 proceeds to an upcare medical transfer protocol 204. The unscheduled medical transfer may be for treatment and transport to an upcare medical facility. A determination 206 is made to confirm if the unscheduled transfer is due to an urgent life-threatening situation. If the caller 114 relays information to the dispatcher 104 that the patient 112 is unconscious and not breathing (or unconscious and breathing is uncertain, or conscious but not breathing where the failure to breathe has been verified), for whatever reason, a determinant code indicating an urgent life threatening condition is generated and a medical transfer is dispatched 208 immediately. The dispatched medical transfer may be a maximum emergency response, which may include resources such as emergency medical technicians, ambulances, paramedics, and other appropriate healthcare providers. The protocol 200 may make further interrogatories to confirm the life threat and provide pre-arrival instructions. The pre-arrival instructions can be tailored to the specific situation and/or condition of the patient, and may include treatment sequence scripts covering, inter alia, cardiac arrest, choking, and childbirth. For example, the treatment sequence scripts may enable the dispatcher to guide the caller in CPR, the Heimlich Maneuver, or emergency childbirth procedures. Typically, the result of properly conveyed (by the dispatcher) and executed (by the caller) instructions is a more viable patient at the time the emergency responders arrive.

The protocol 200 may guide the dispatcher through a secondary interrogation 210 focusing on the patient's medical condition or chief complaint. The protocol 204 may present a pre-scripted interrogation to enable a more orderly and detailed understanding of the patient so that the pre-hospital care provided by the emergency responders is appropriate for the severity of the patient's condition. The pre-scripted interrogation may include preprogrammed inquiries focused on gathering information relating to the chief complaint.

The secondary interrogation 210 may include instructions for the dispatcher 104 to remain on the telephone with the caller 114 to provide post-dispatch instructions regarding what to do, and what not to do, prior to the arrival of the emergency responders. The post-dispatch instructions help to prepare the patient 112 for, and to expedite, the emergency responders' work at the scene. Post-dispatch instructions may include preparing the patient for transport, prompting the collection of pertinent documents that will accompany the patient, and the relaying of any other pertinent information related to the transport.

If the patient's medical condition or chief complaint is determined 206 to be non-life threatening, then the protocol 200 continues with additional non-life threatening interrogations 212 or preprogrammed inquires to determine the nature of the medical condition, generate a determinate code, and dispatch 214 a medical transfer with an appropriate medical vehicle and trained medical personnel. In either dispatch 208 or 214, the medical transfer protocol 110 generates a determinant code and billing code.

If Case Entry 202 determines that the medical transfer is scheduled or routine and the patient 112 does not have an urgent medical need, then the protocol 200 routes to a schedule medical transfer protocol 216. The schedule medical transfer protocol 216 includes an interrogation 218 or preprogrammed inquires to query the caller 114 for the destination, nature of medical treatment, time and date of transfer, type of medical treatment required, type of medicine required, whether the patient 112 can walk or respond, and the like. The interrogation 218 may further inquire as to the weight of the patient 112 in order to determine if the medical vehicle must include mechanical lift assistance. Based on this information, the medical transfer protocol 110 generates a determinant code, billing code, and dispatches 220 a medical transfer with a suitably equipped medical vehicle at the scheduled time.

Case Entry 202 may determine that the medical transfer relates to mental health treatment, routine and scheduled or non-scheduled. If the patient 112 does not have an urgent medical need, then the protocol 200 routes to a mental health transfer protocol 222. The mental health protocol 222 includes interrogatories or preprogrammed inquires 224 to query the caller 114 for the destination, nature of mental health treatment, time and date of transfer, required medical treatment, medical equipment, or medicine during the transfer, whether the patient 112 can walk or respond, weight of the patient, and whether the patient is a threat to himself/herself or anyone else. Based on this information, the mental health transfer protocol 222 generates a determinant code, billing code, and dispatches 226 an appropriate medical transfer at the scheduled time.

If during interrogatories 218, 224, the dispatcher 104 receives information from the caller 114 to indicate any acute medical problems that are more significant this the current mental health issue, the protocol 200 may shunt directly to a determination 206 to verify the patient's condition. The protocol 200 at determination 206 guides the dispatcher 104 to gather information from the caller 114 to enable the dispatcher 104 to ascertain the patient's medical condition or chief complaint. The protocol 200 may then proceed as previously discussed above.

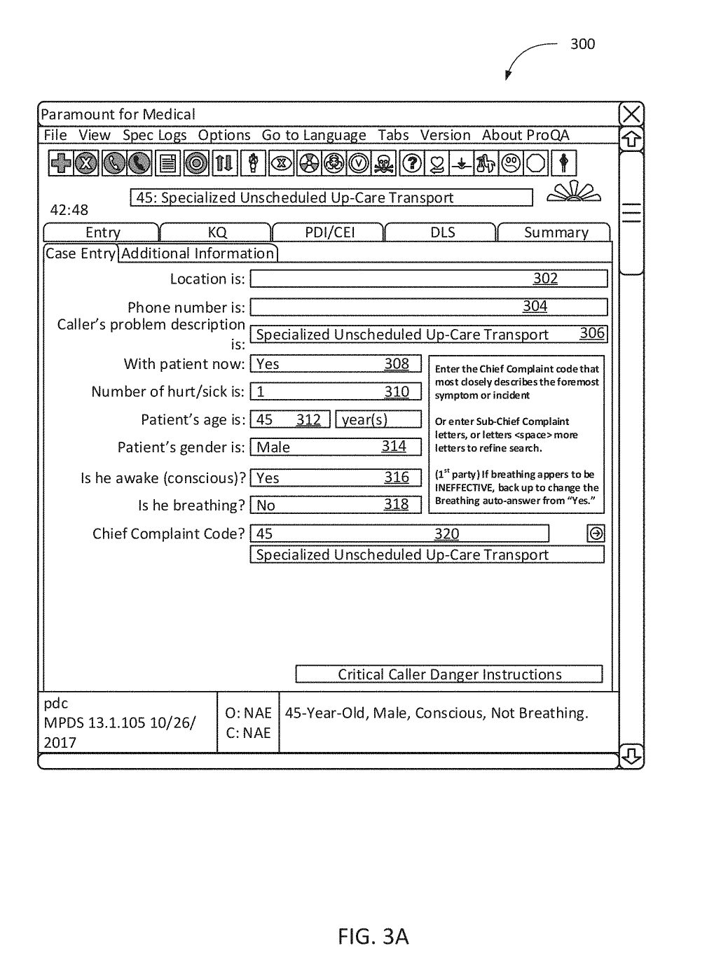

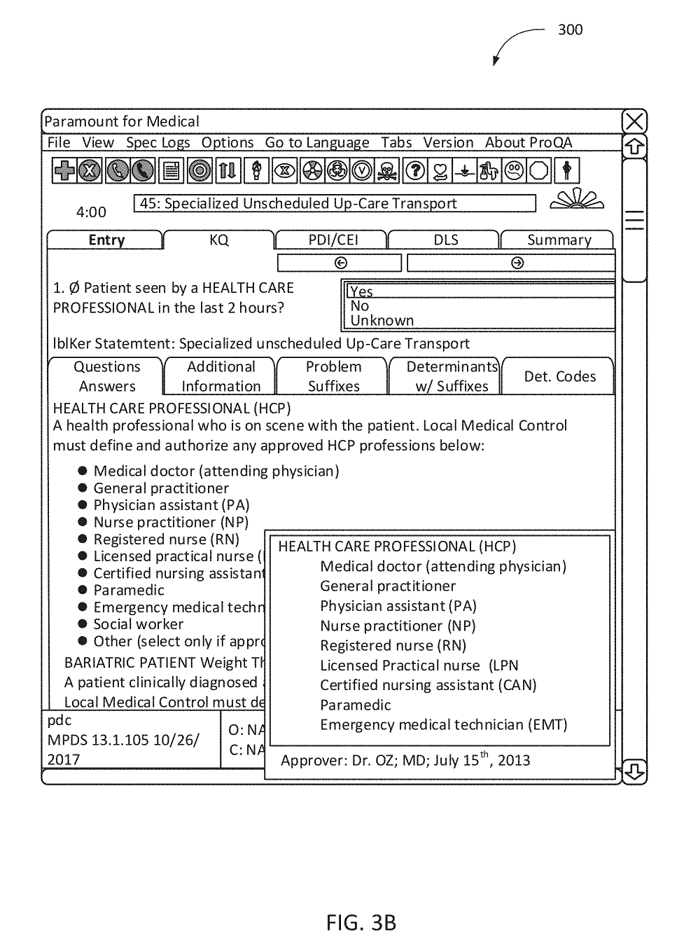

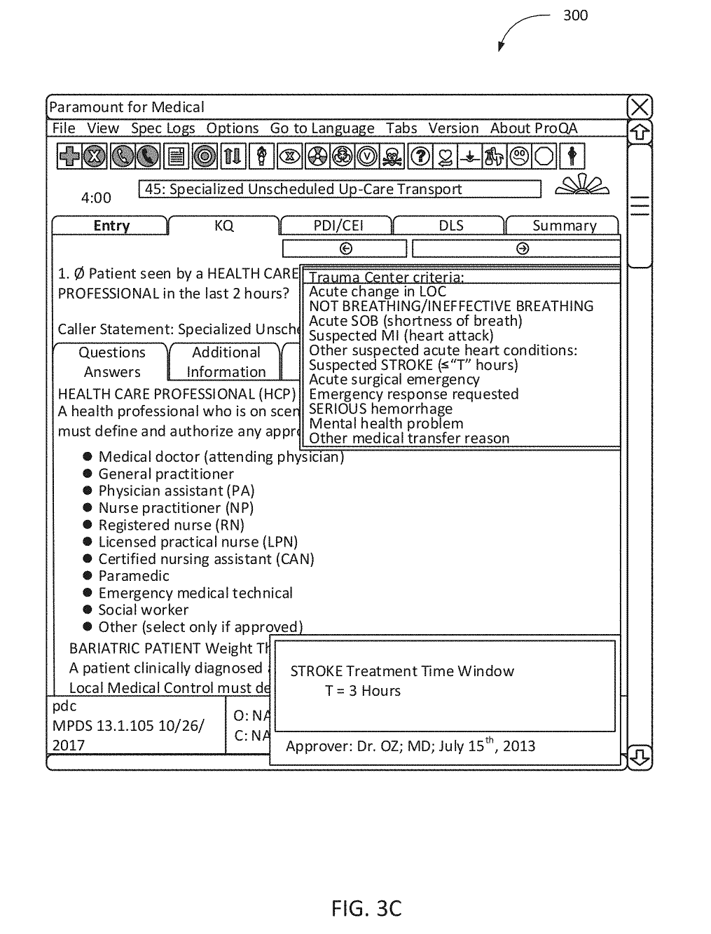

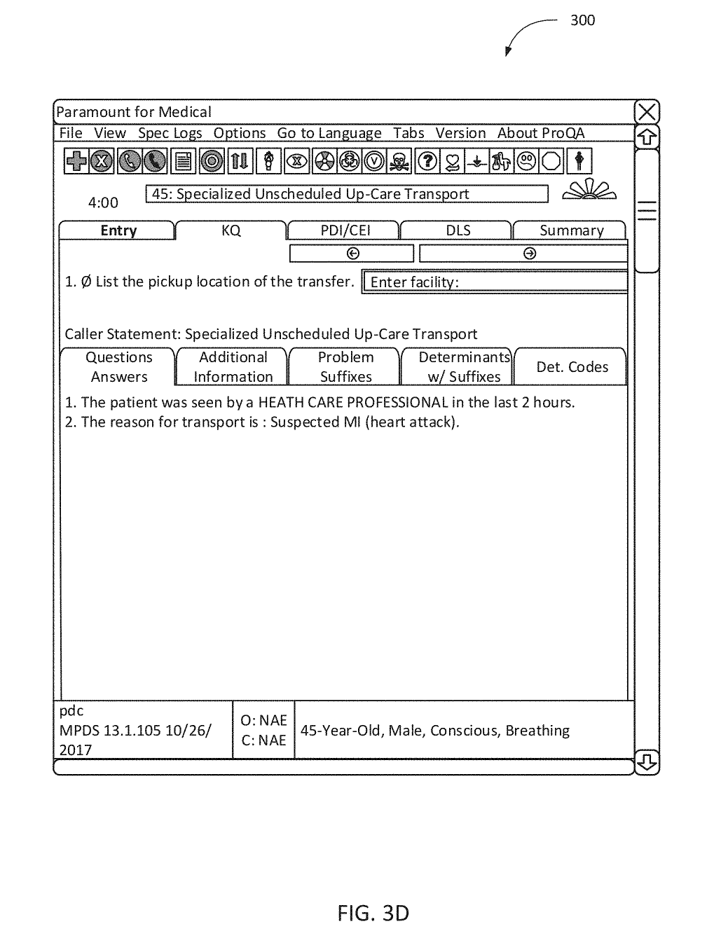

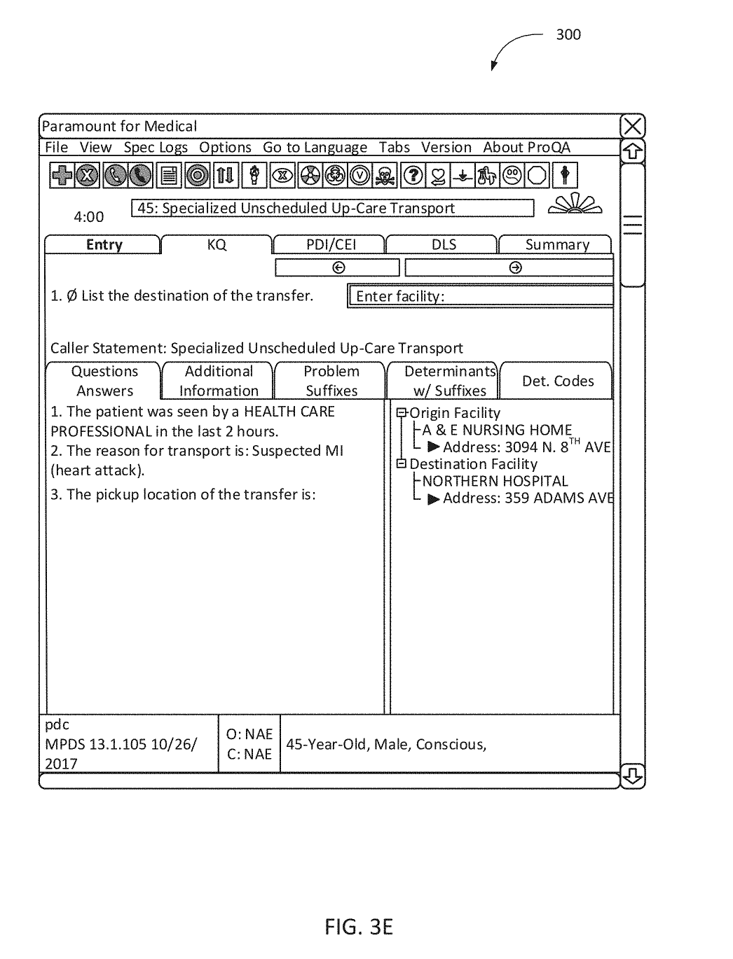

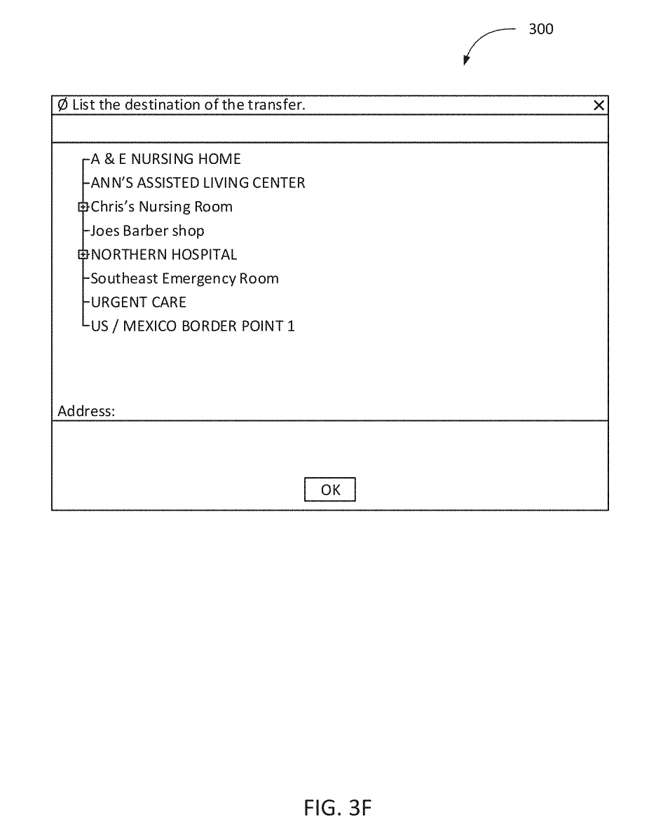

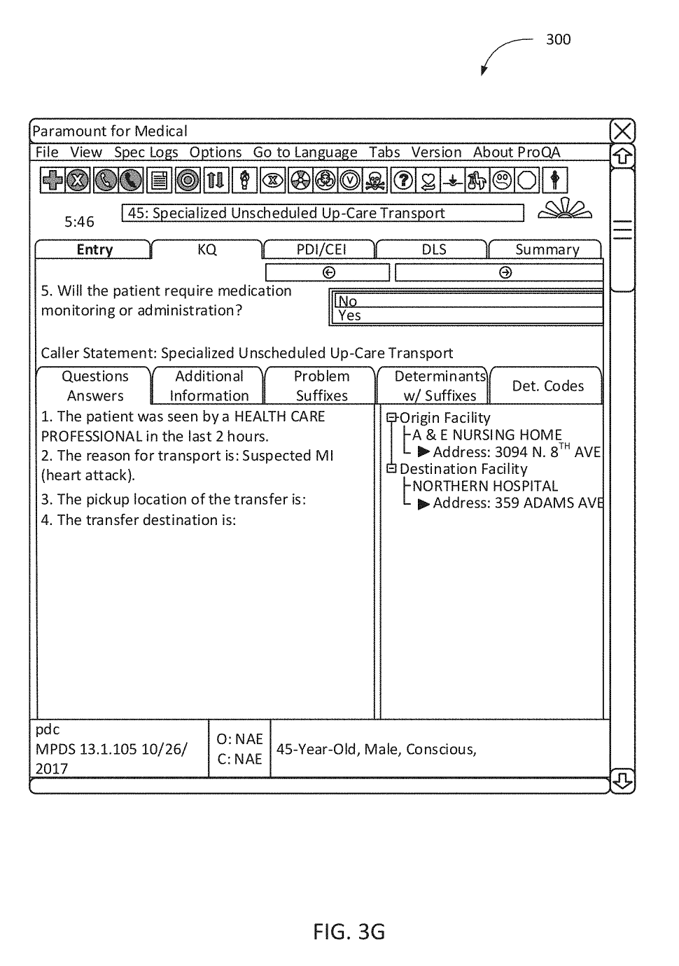

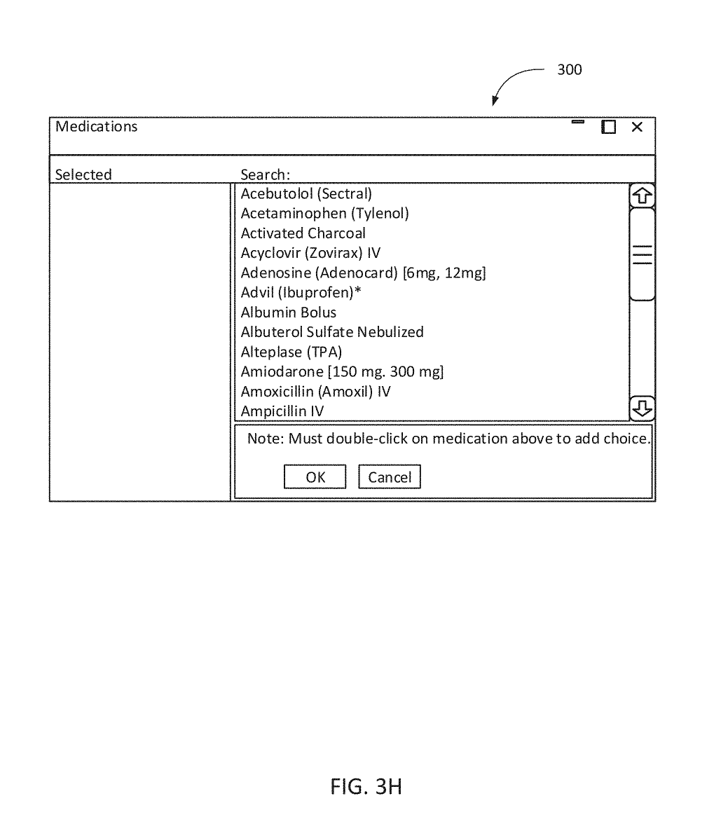

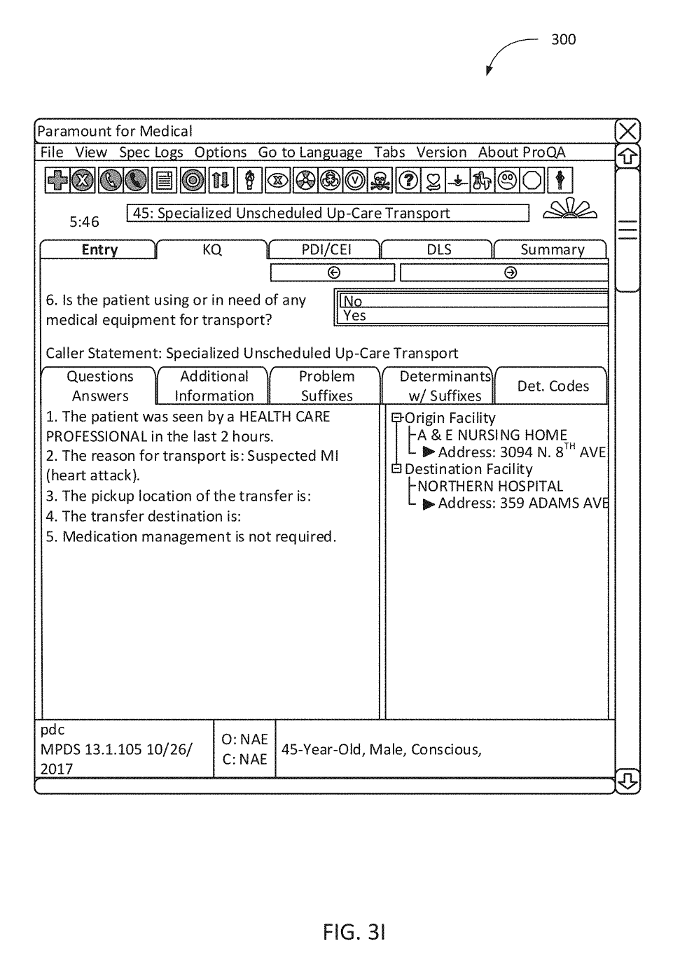

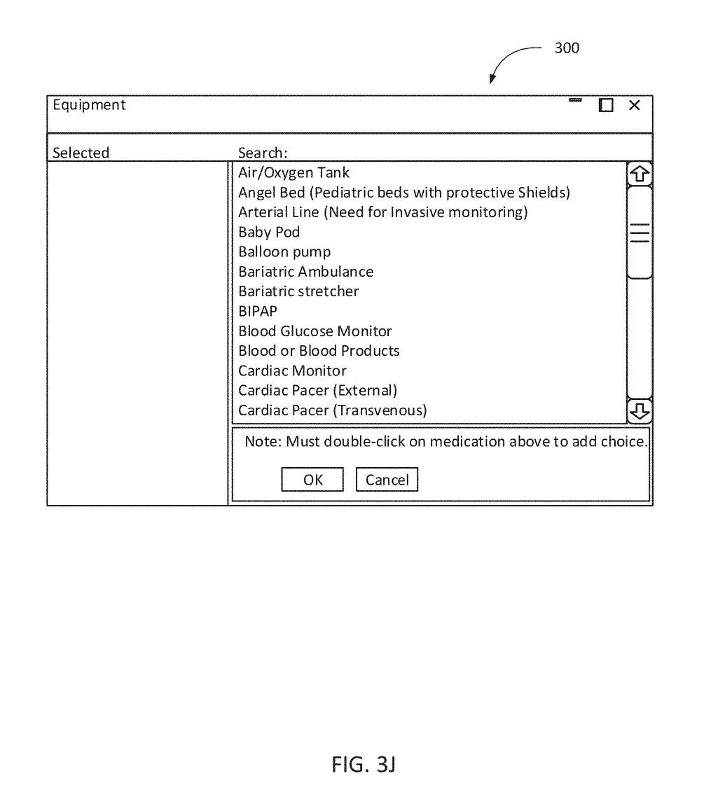

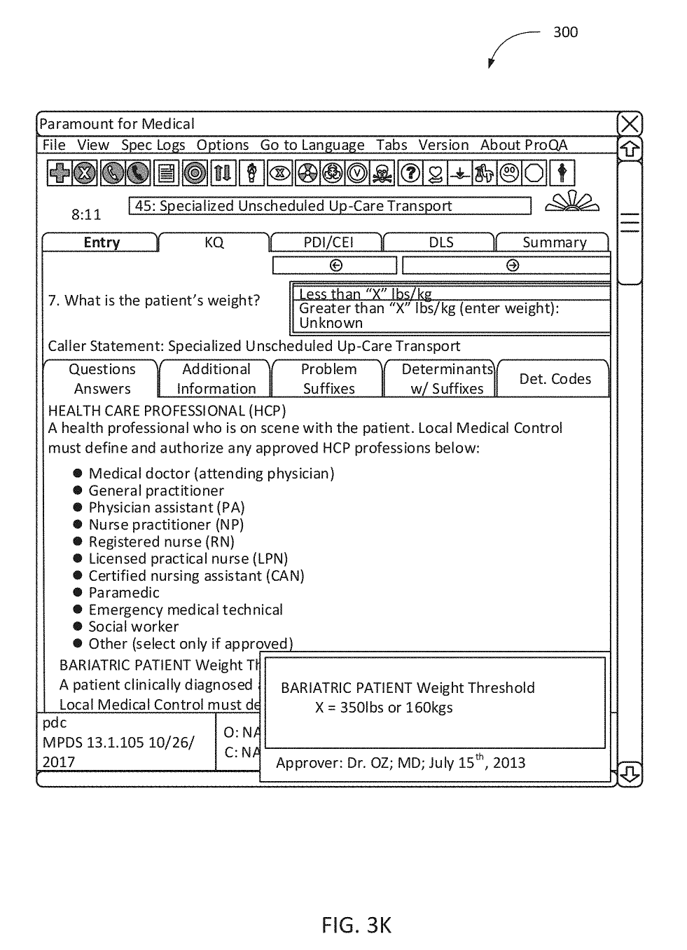

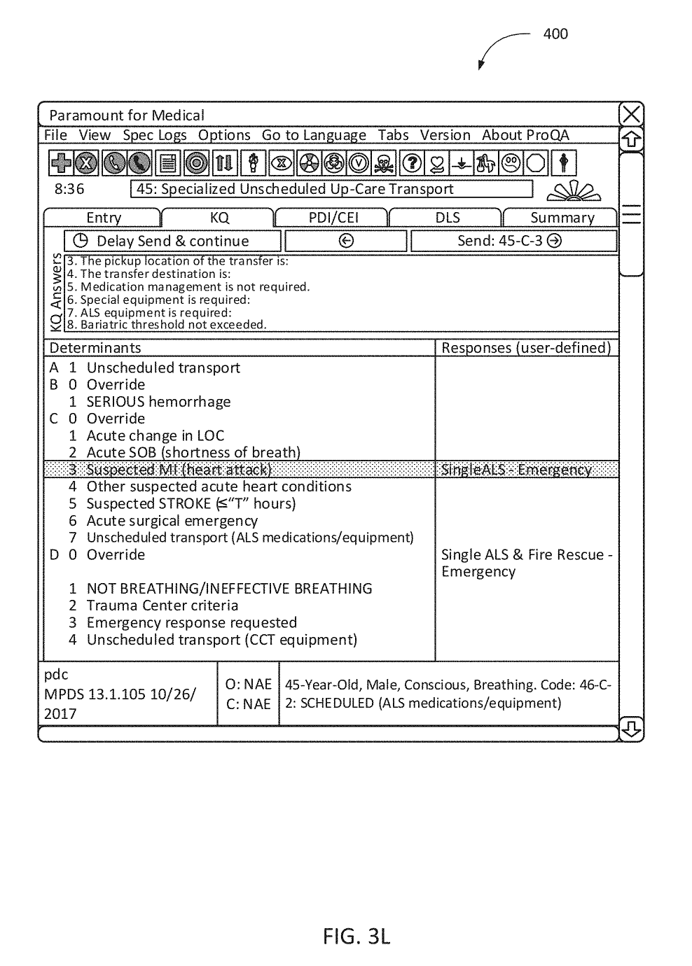

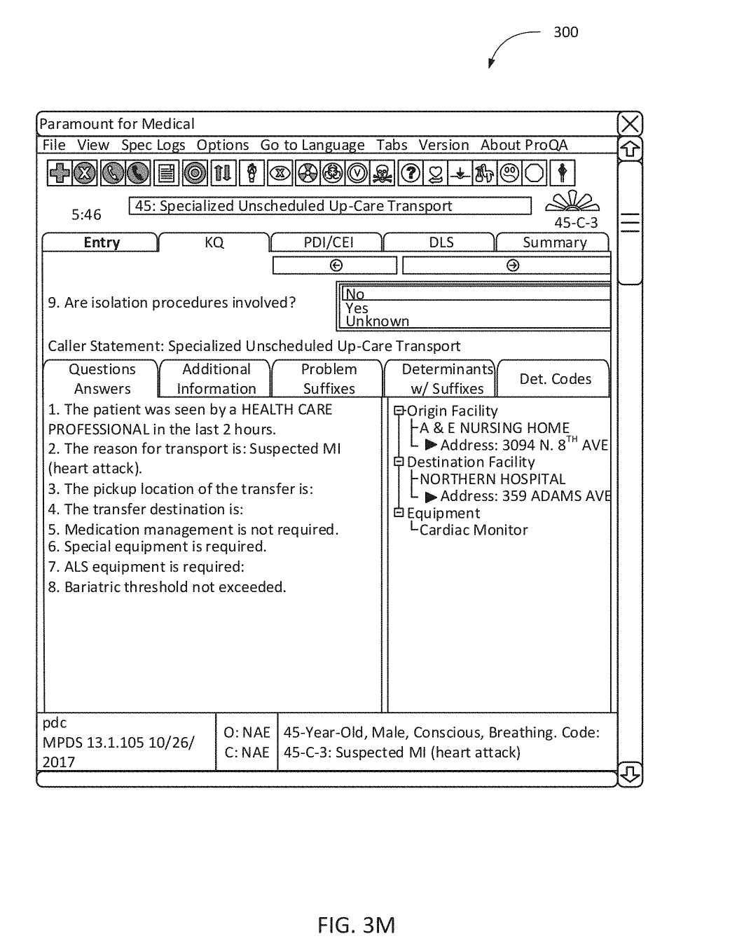

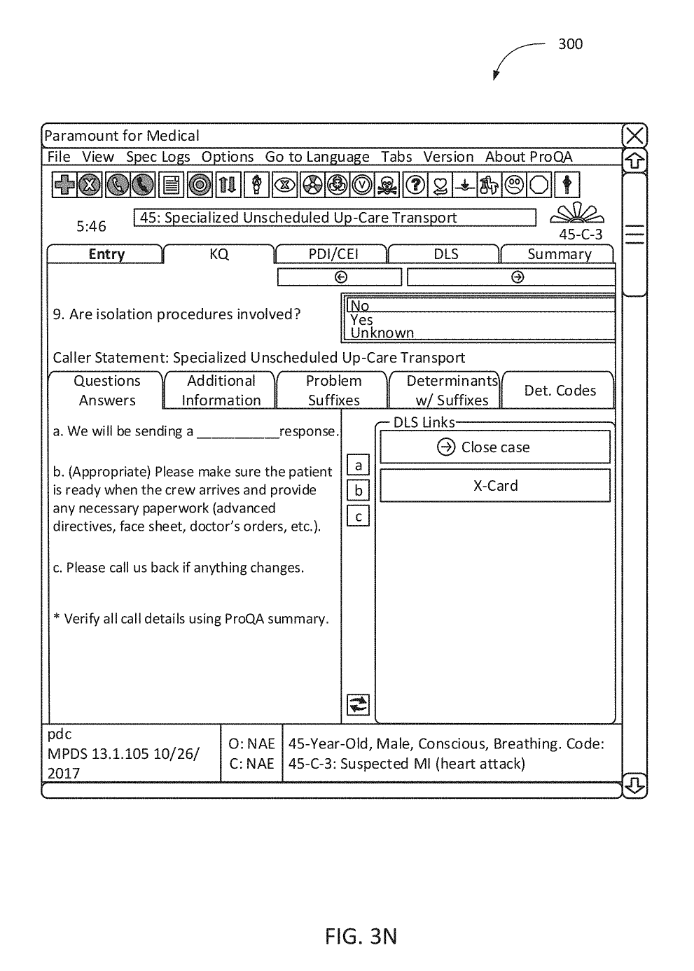

Referring to FIGS. 3A-3N, embodiments of a user interface 300 are shown for the medial transfer protocol processing an unscheduled medical transfer. In FIG. 3A, the user interface 300 provides input fields for the location of the patient 302, the phone number of the caller 304, the caller's problem description 306, whether the caller is with the patient 308, how many are hurt or sick 310, the patient's age 312, the patient's gender 314, if the patient is conscious 316, if the patient is breathing 318, and a chief compliant code 320. A chief complaint code may be used in industry practice and known to the dispatcher 104 to expedite the medical transfer. In 318, the patient is identified as breathing. The medical transfer is confirmed in 320 as being unscheduled and requiring transport to an up-care facility.

In FIG. 3B, the medical transfer protocol confirms that the patient has been seen by a healthcare professional.

In FIG. 3C, the call-taker selects the most appropriate medical complaint, which is a decision-tree driver.

In FIGS. 3D and 3E, the protocol allows the call-taker to select both a pick-up and drop off location from a preprogrammed list in FIG. 3F.

In FIG. 3G, the protocol continues with interrogation relating to the administration or monitoring of medications from a preprogrammed list in FIG. 3H which is also a decision-tree driver.

In FIG. 3I, the protocol continues with interrogation relating to the use of medical equipment from a preprogrammed list in FIG. 3J which is also a decision-tree driver.

In FIG. 3K, the protocol generates a question relating the weight of the patient allowing agencies to mitigate bariatric patients.

In FIG. 3L, the protocol offers a dispatch point with a recommended determinant code based on the callers answers to the interrogation questions.

In FIG. 3M, after the dispatcher accepts the determinant code the call-taker asks additional questions that are informational but not determinant drivers. These questions are locally defined.

In FIG. 3N, post-dispatch instructions may include preparing the patient for transport, prompting the collection of pertinent documents that will accompany the patient, and the relaying of any other pertinent information related to the transport.

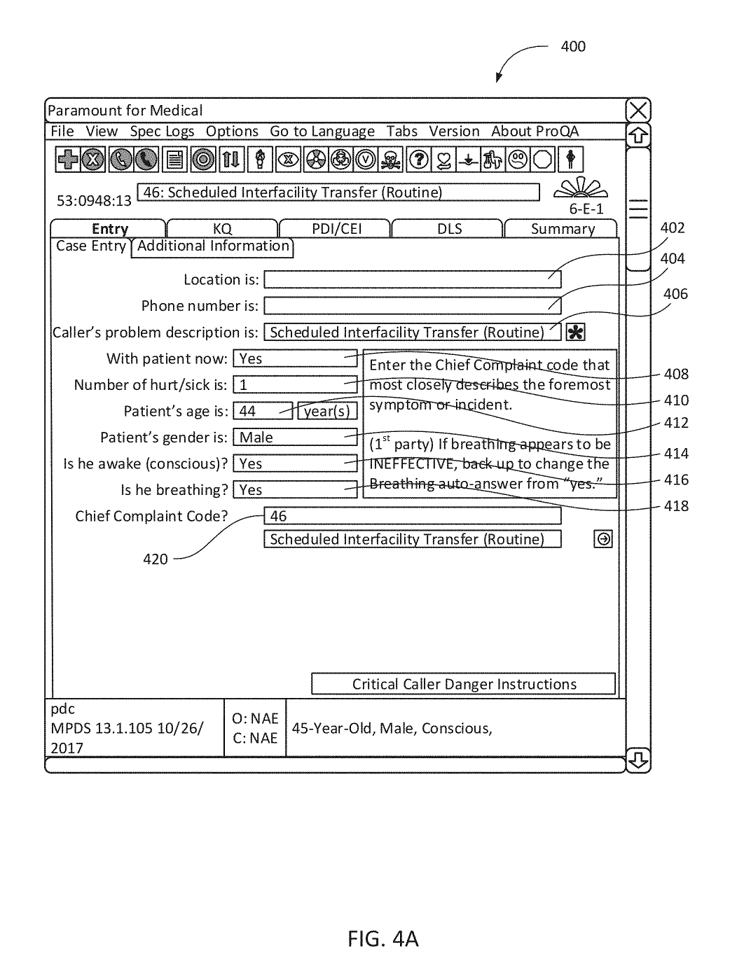

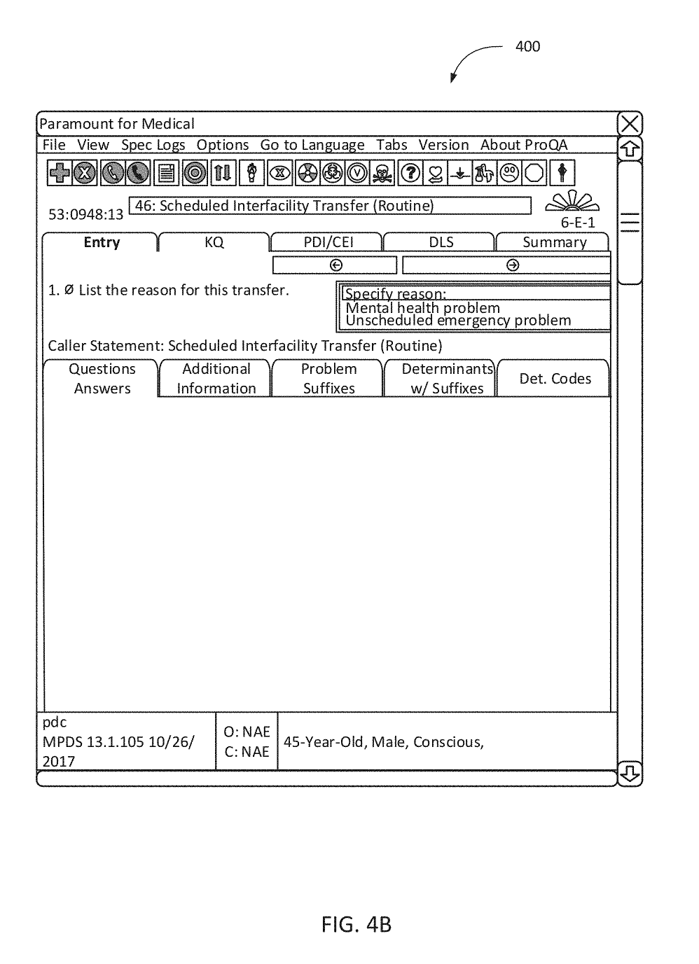

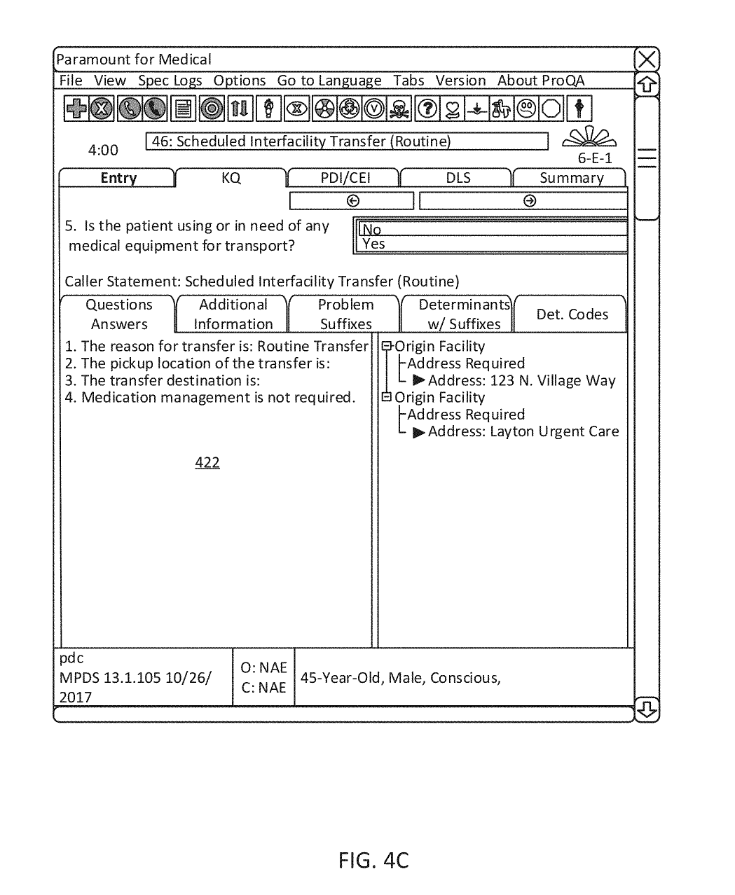

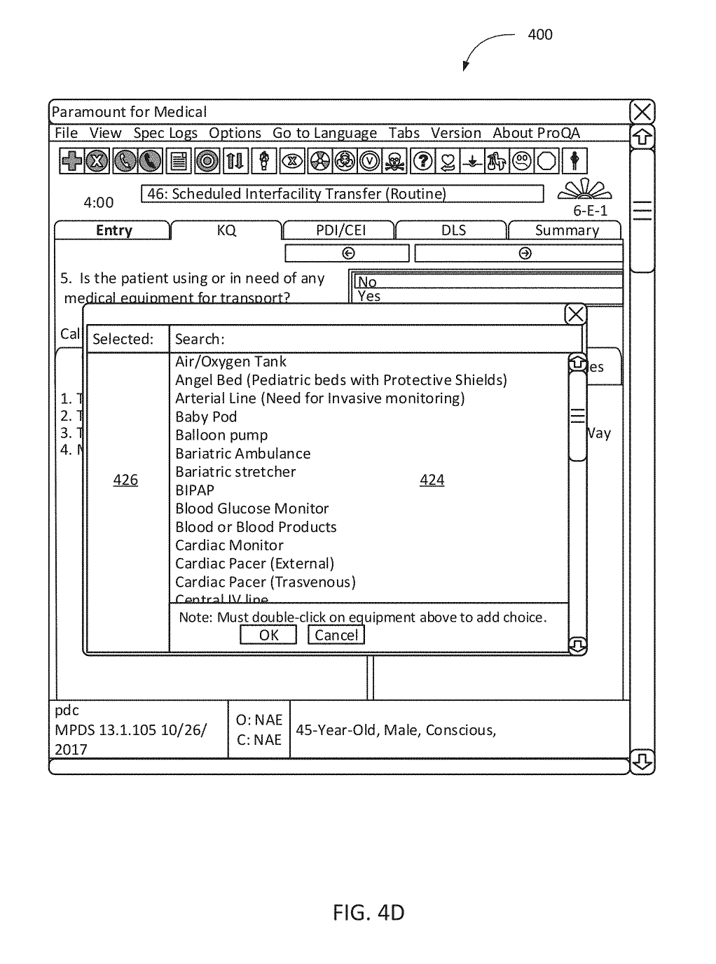

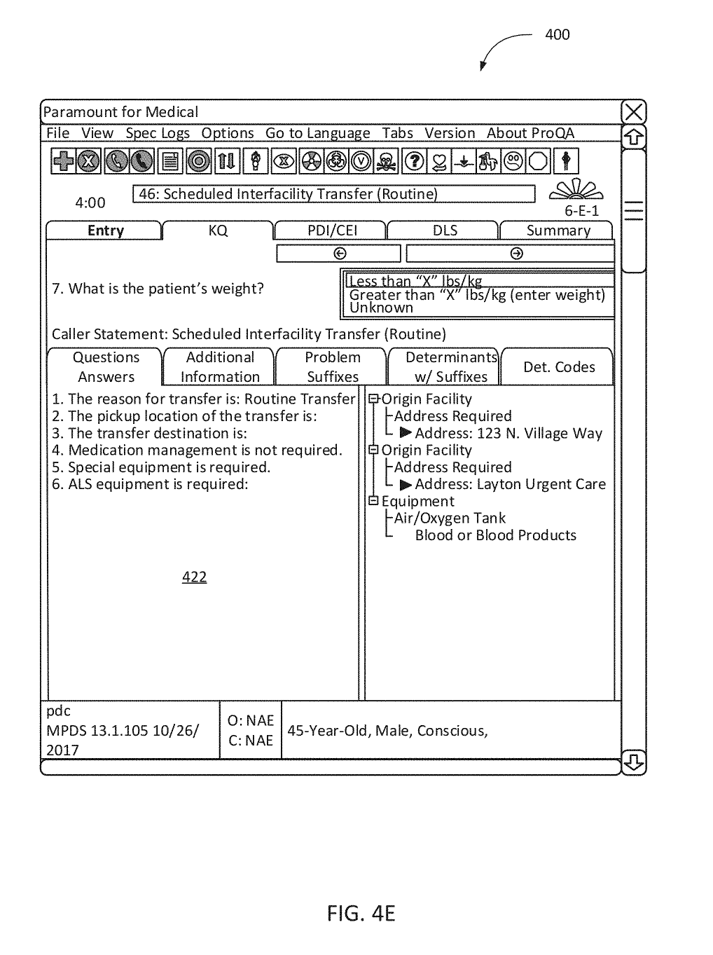

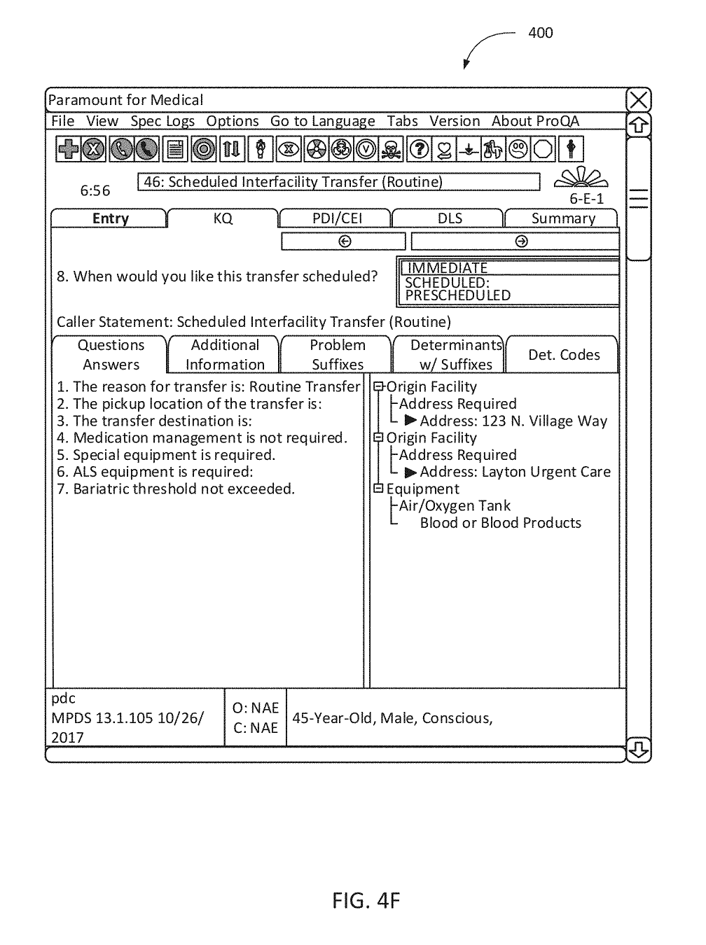

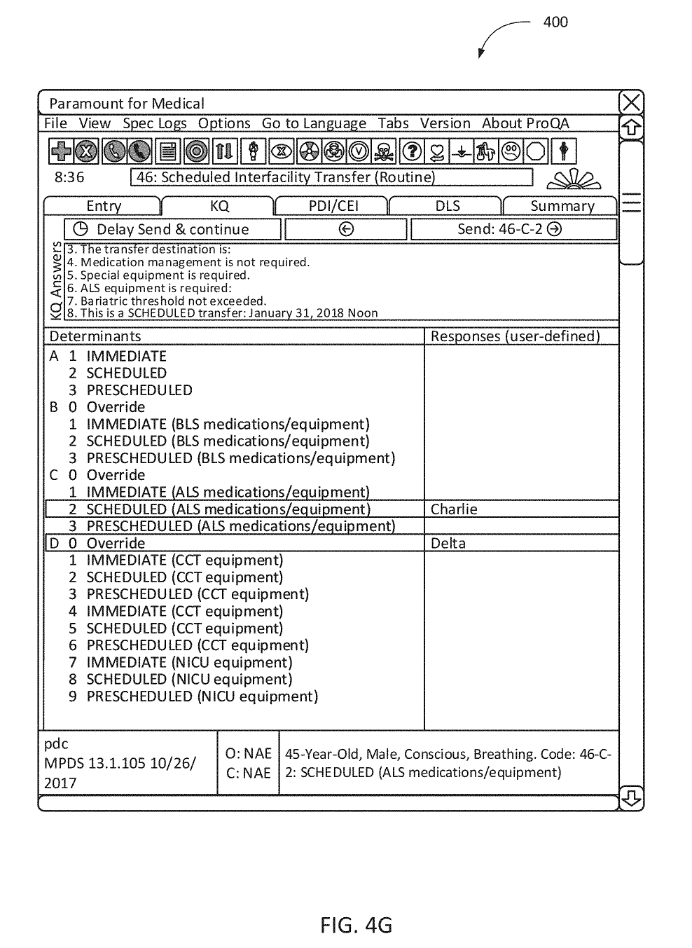

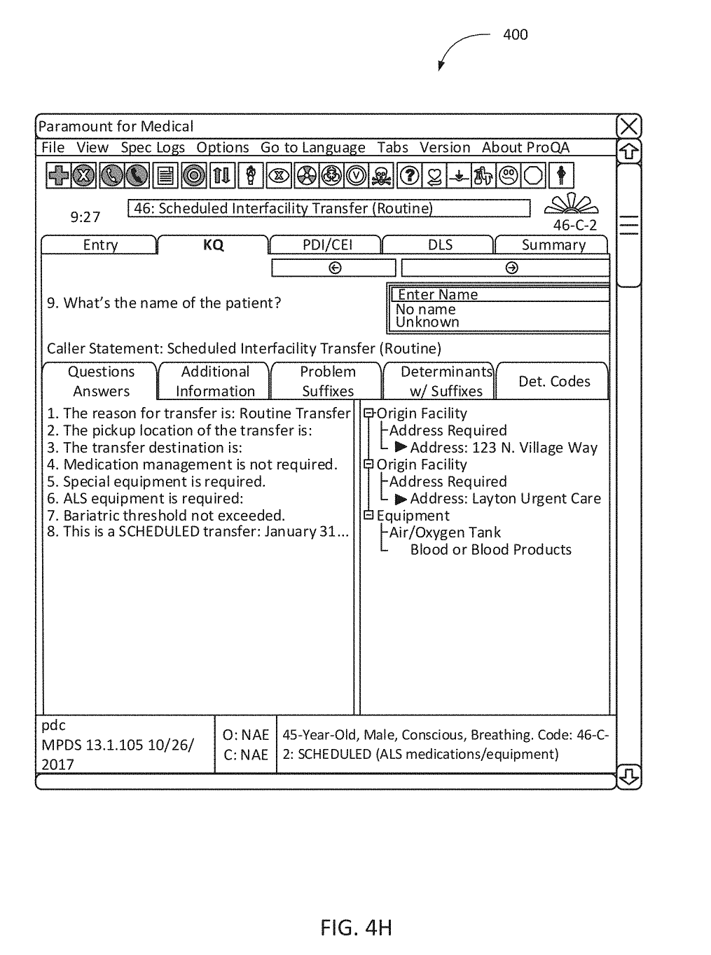

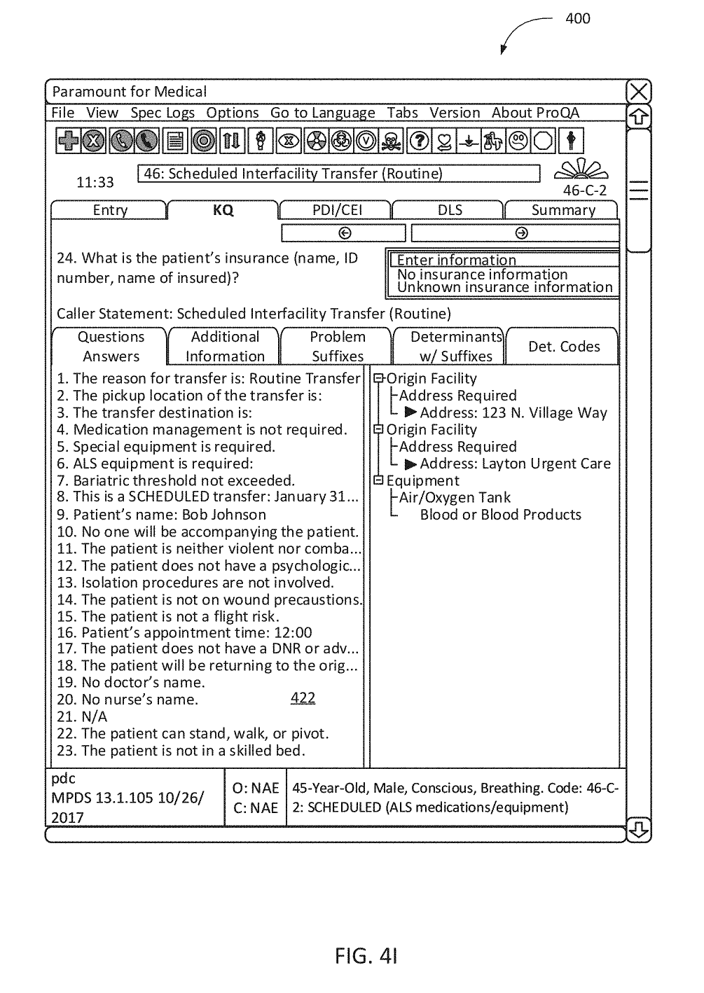

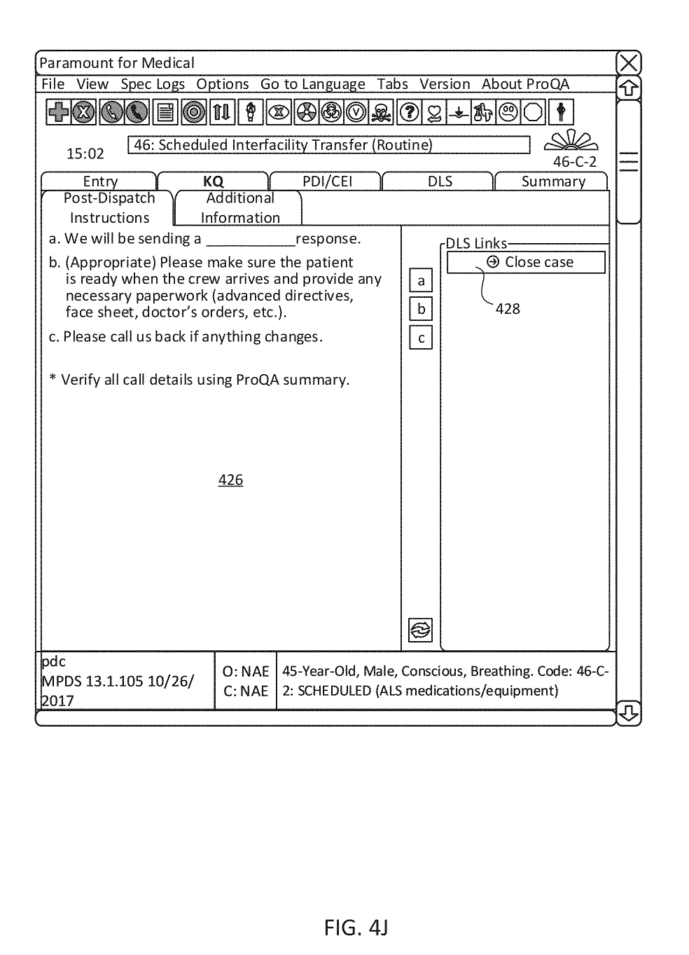

FIGS. 4A-4J represent an embodiment of a user interface illustrating the medical transfer protocol in processing a routine medical transfer request.

In FIG. 4A, the dispatcher initiates the medical transfer protocol to begin the case entry for a scheduled medical transfer. The medical transfer may be a routine transfer from one medical facility to another. In one embodiment, the user interface 400 queries the dispatcher for the location 402 of the patient, a contact telephone number 404, the caller's problem description 406, if the caller is presently with the patient 408, the number of patients 410, the patient age 412, the patient gender 414, if the patient is conscious 416, if the patient is breathing 418, and a chief complaint code 420. As shown, a chief complaint code is entered which corresponds to a scheduled interfacility transfer (routine). The caller is often a trained employee at a medical facility or may even be at the patient's resident.

In FIG. 4B, the user interface 400 queries for a reason for the transfer. As the chief complaint code was previously established as a routine transfer, the default is that of a routine transfer.

In FIG. 4C, the user interface 400 queries about medical equipment needed for the transfer. The user interface 400 has previously queried about medicine management and a caller statement field 422 indicates that this has been answered and medicine management is not needed. As such, the transfer vehicle will not be required to bring requested medicine. The caller statement field 422 further notes a destination facility address which the dispatcher has entered based on an earlier query.

In FIG. 4D, the dispatcher has acknowledged the need for medical equipment. The user interface 400 populates an equipment field 424 with a list of numerous equipment options. The dispatcher may select one or more of the equipment options which are then populated in field 426.

In FIG. 4E, the user interface prompts for the patient's weight. If the patient exceeds a threshold, then the medical vehicle arrives with the needed assist equipment. In the caller statement field 422, special equipment is noted as being required. The chosen equipment is Air/Oxygen Tank and Blood or Blood Products.

In FIG. 4F, the user interface prompts for the transfer schedule. As shown, in one embodiment, the dispatcher may choose from: immediate, scheduled, or prescheduled. In addition to choosing one of the three options, the dispatcher may select the date and time.

In FIG. 4G, the user interface lists the scheduled transfer date and time and the determinate code of Charlie which indicates a moderate priority.

In FIG. 4H, the medical transfer protocol prompts for the patient's name.

In FIG. 4I, the medical transfer protocol prompts for the patient's insurance information. The caller statement field 422 indicates that the caller has previously responded to protocol queries and provided that no one will accompany the patient, the patient does not have a psychological disorder, isolation procedures are not involved, there are no wound precautions, the patient is not a flight risk, the patient's appointment time, the patient does not have a Do Not Resuscitate directive, the patient will be returning to the originating facility, the doctor's name, the nurse's name, whether the patient can stand, walk, or pivot, and the patient is not in a skilled bed.

In FIG. 4J, the user interface provides post-dispatch instructions in the field 426 for the dispatcher to read to the caller. The user interface provides an option 428 to close the case. When the medical transfer is scheduled, a billing code is also generated based on the transfer vehicle, requested medical equipment, requested medicine, date and time of transfer, origination and destination, and any other entered factors. The billing code is then electronically saved with the record of the medical transfer.

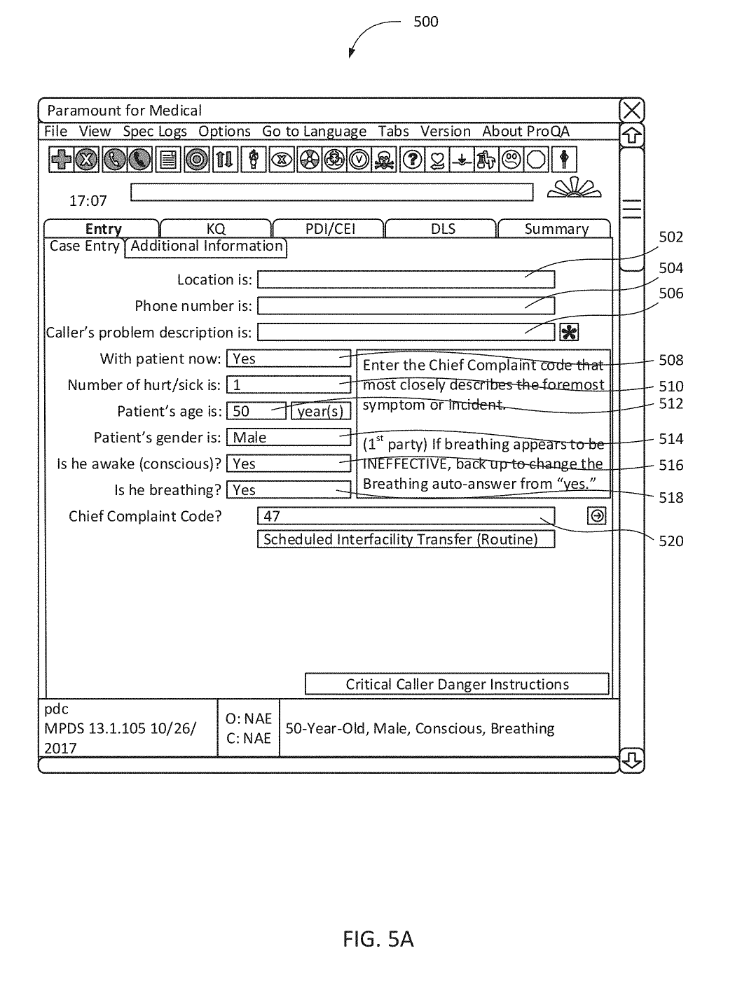

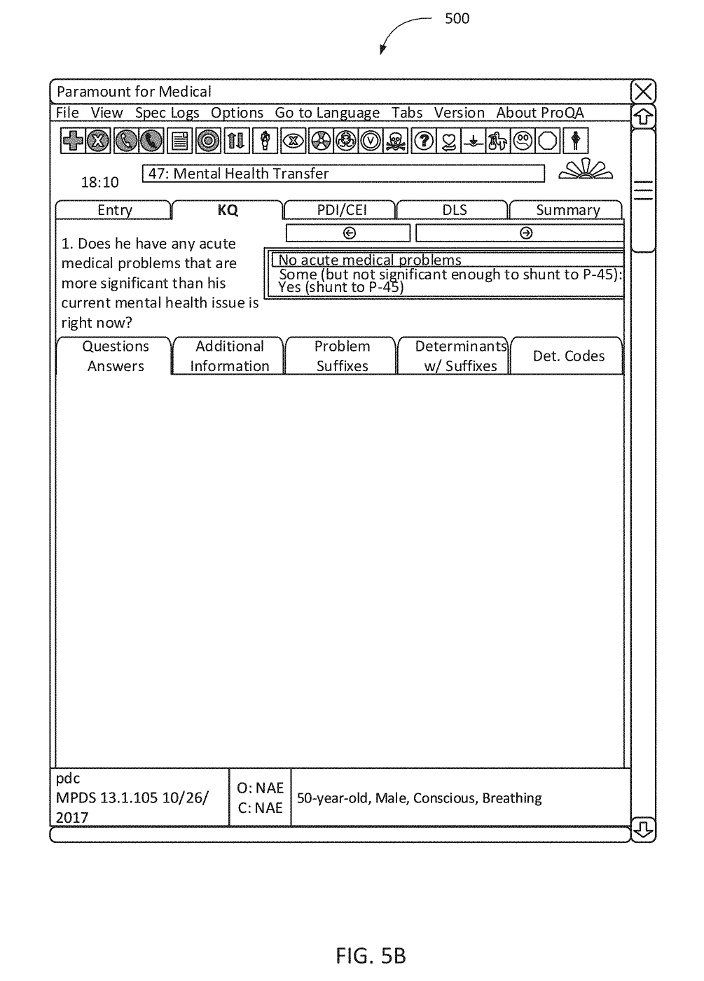

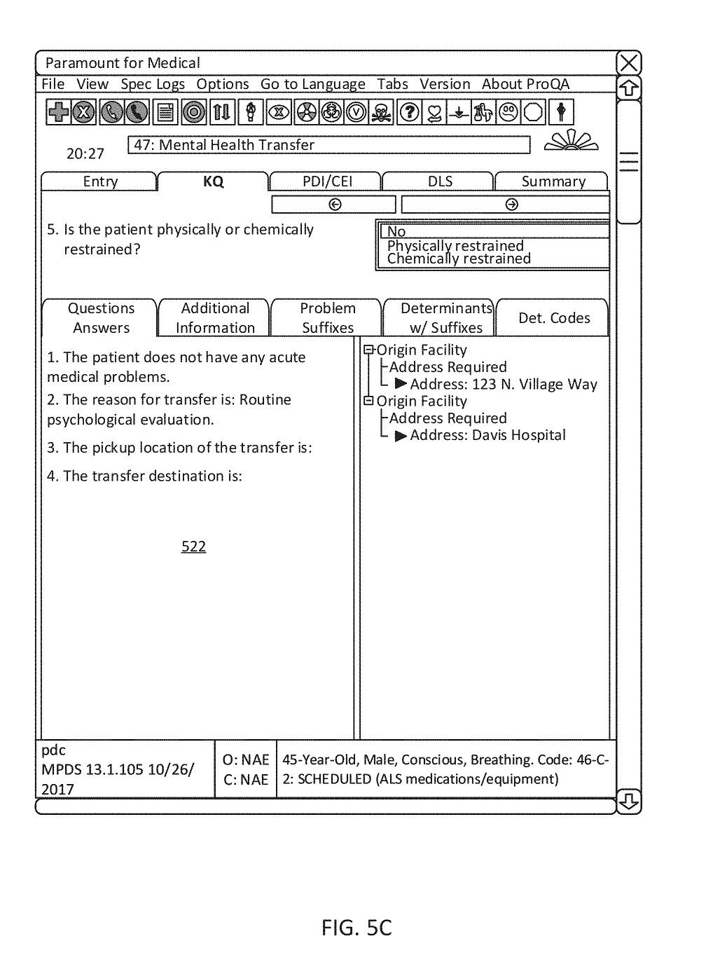

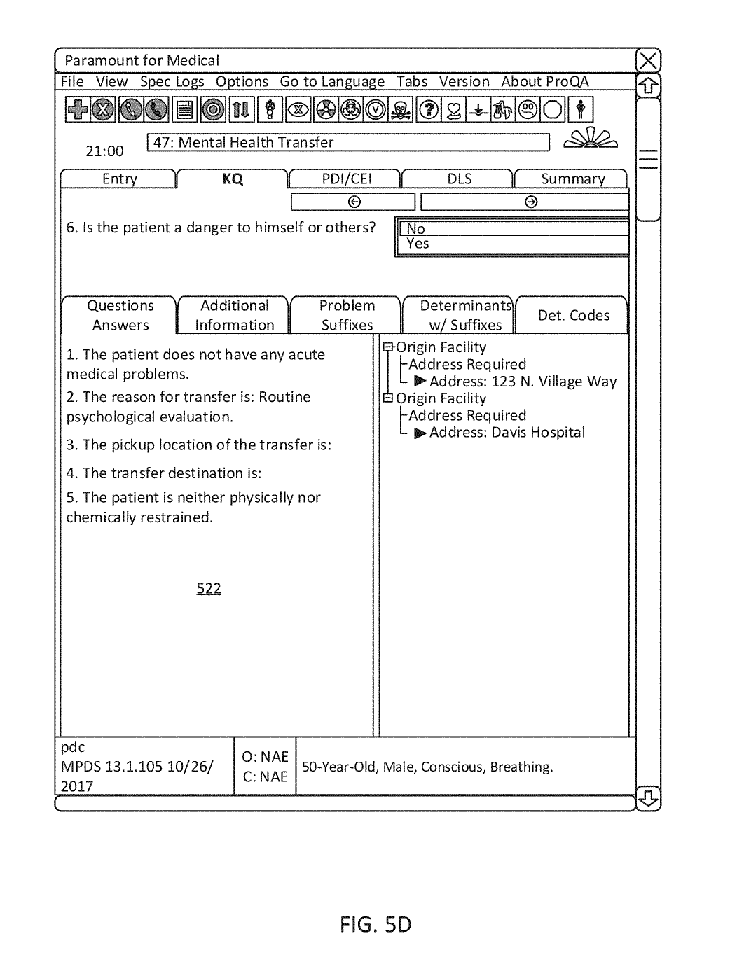

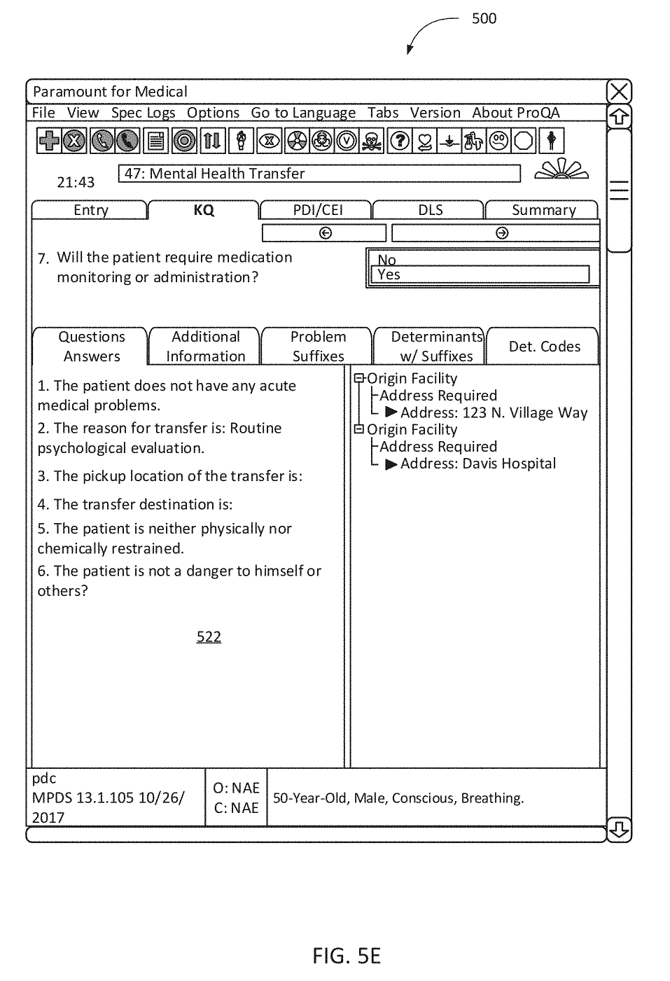

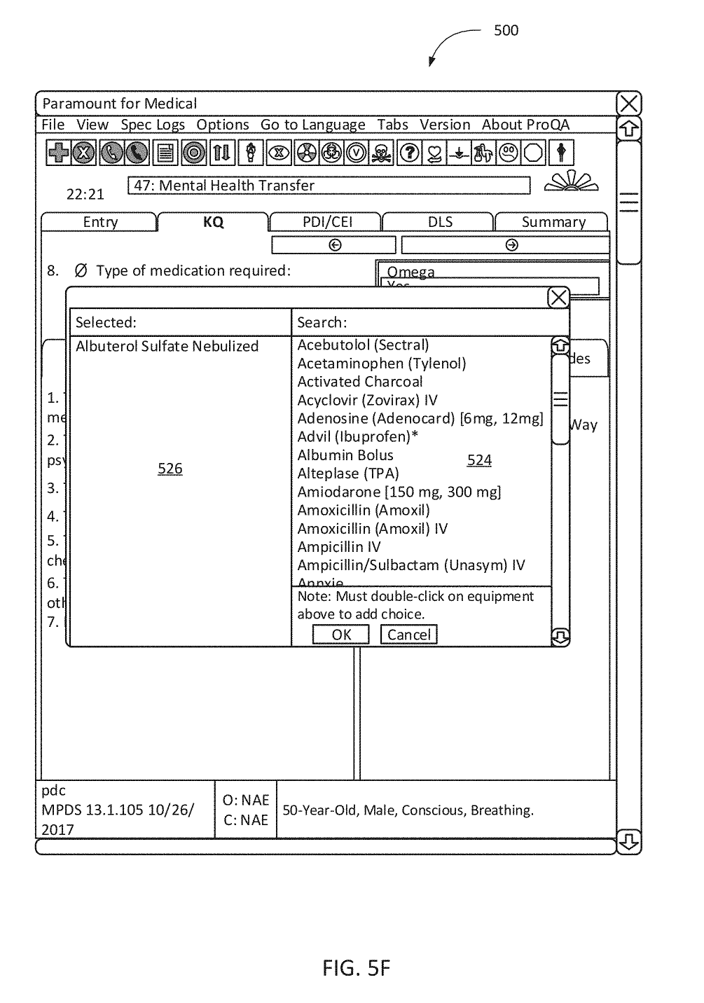

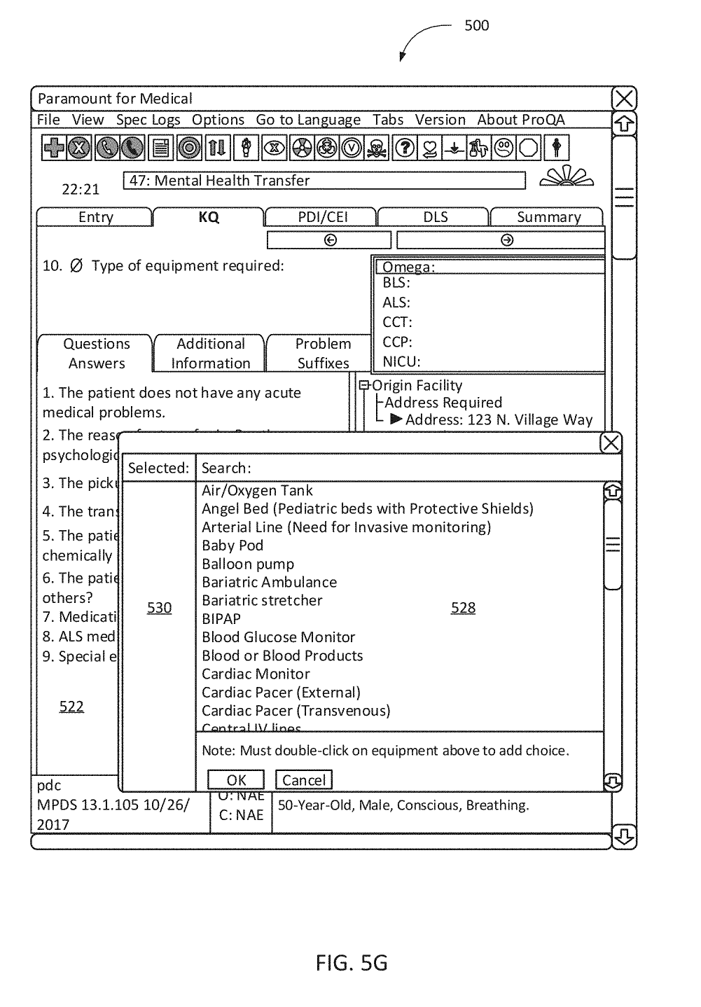

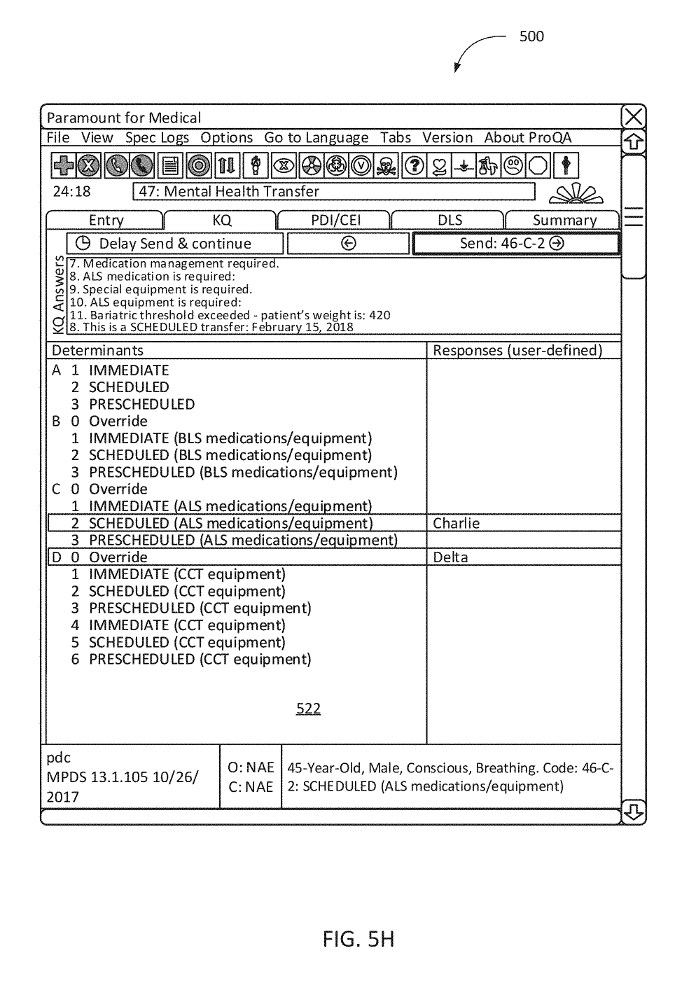

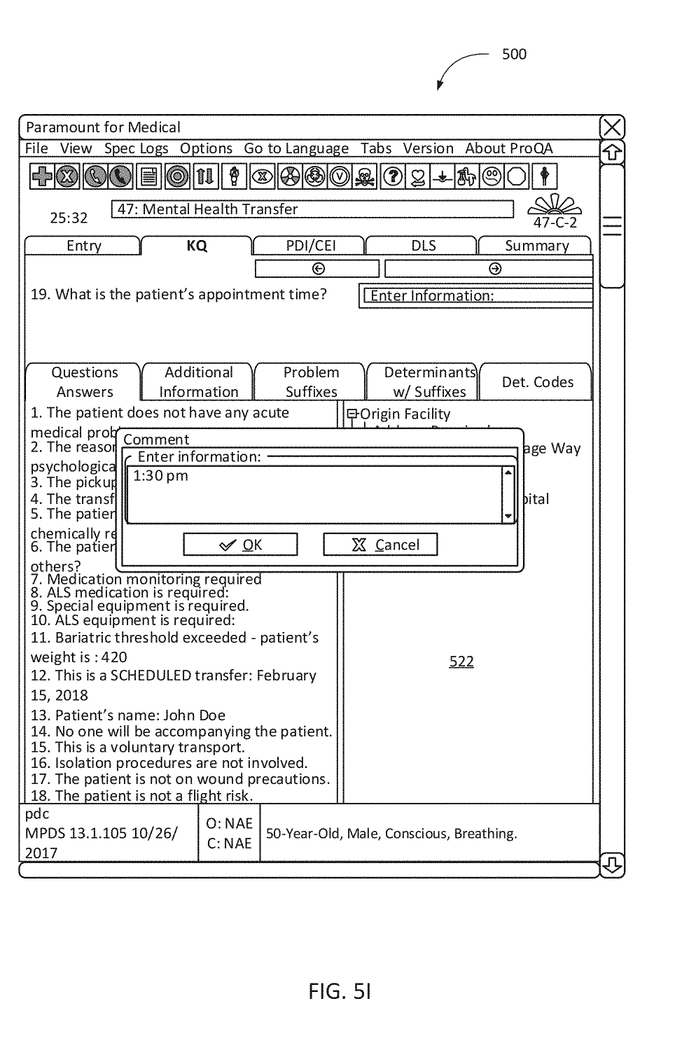

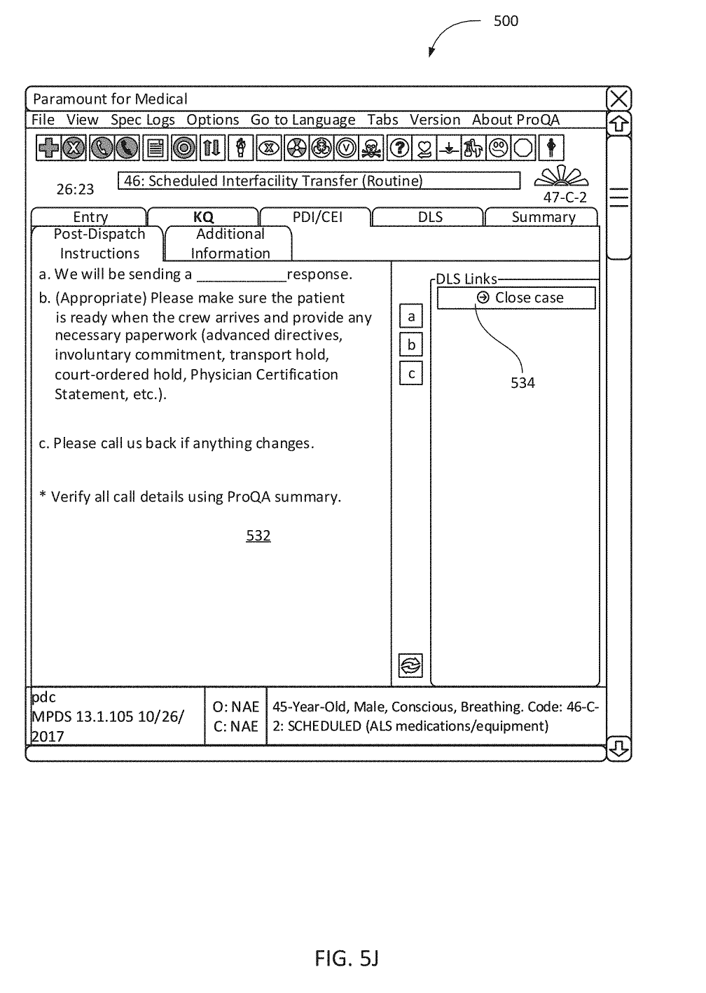

FIGS. 5A-5J represent an embodiment of a user interface illustrating the medical transfer protocol in processing a mental health transfer request.

In FIG. 5A, the dispatcher initiates the medical transfer protocol to begin the case entry for a mental health transfer. The transfer may be for counseling, therapy, medicinal administration, or other medical attention. Similar to the disclosure above, the user interface 500 queries the dispatcher for the location 502 of the patient, a contact telephone number 504, the caller's problem description 506, if the caller is presently with the patient 508, the number of patients 510, the patient age 512, the patient gender 514, if the patient is conscious 516, if the patient is breathing 518, and a chief complaint code 520. As shown, a chief complaint code is entered which corresponds to a mental health transfer. As can be appreciated, a mental health transfer involves additional considerations not needed in other medical transfers.

In FIG. 5B, the medical transfer protocol queries as to whether the patient has any acute medical conditions that are more significant than the current mental health issue. If so, the medical transfer protocol shunts to an unscheduled medical transfer such as that represented in FIGS. 3A-3I. A scheduled medical transfer, such as that represented in FIGS. 4A-4J, may also include an option to shunt to an unscheduled medical transfer.

In FIG. 5C, the caller statement field 522 notes that there are no acute medical problems, the reason for the transfer, the pickup location, and the transfer destination. The medical transfer protocol queries as to whether the patient needs to be physically or chemically restrained. If so, the protocol will require a medical vehicle with the corresponding equipment.

In FIG. 5D, the caller statement field 522 notes that the patient does not need to be physically or chemically restrained. The medical transfer protocol then queries as to whether the patient is a danger to the patient or to others. This information may be sent to the medical personnel providing the transfer.

In FIG. 5E, the caller statement field 522 notes that the patient is not a danger to the patient or others. The medical transfer protocol queries as to whether the patient requires medication monitoring or administration.

In FIG. 5F, a medications field 524 is opened in response to an affirmation that the patient requires medication. The dispatcher may then select one or more medicines from the provided list. The selected medicines are then populated in the field 526. The medical transfer protocol will require a medical vehicle that has the selected medicine in its inventory. This information may also be provided to the medical transfer personnel.

In FIG. 5G, the caller statement field 522 notes that medical equipment is required in response to a previous caller query. The medical transfer protocol queries for the required equipment and provides an equipment field 528 listing various equipment options. The dispatcher may select one or more equipment options which are then populated in field 530. The medical transfer protocol will require a medical transfer vehicle with the corresponding selected equipment.

In FIG. 5H, the caller statement field 522 of the user interface lists that the patient has a weight that exceeds a bariatric threshold in response to a previous query. As such, the medical transfer vehicle will require corresponding equipment. The caller statement field 522 further notes the date of the scheduled transfer in response to a previous query. The user interface notes an assigned determinant code of Charlie which indicates a moderate priority. As discussed, above the determinant code is assigned by the determinant code calculator automatically (i.e., without user intervention).

In FIG. 5I, the caller statement field 522 lists the patient's name, that no one will accompany the patient, that the transfer is a voluntary transport, that isolation procedures are not involved, that the patient is not on wound precautions, and that the patient is not a flight risk, in response to previous medical transfer protocol queries. The medical transfer protocol queries for an appointment time, the dispatcher enters the time, and the user interface displays a confirmation of the time.

In FIG. 5J, the user interface provides post-dispatch instructions in the field 532 for the dispatcher to read to the caller. The user interface provides an option 534 to close the case. When the mental health transfer is scheduled, a billing code is also generated based on the transfer vehicle, requested medical equipment, requested medicine, date and time of transfer, origination and destination, and any other entered factors. The billing code is then electronically saved with the record of the medical transfer.

As disclosed, a medical transfer protocol objectively selects appropriate resources based on the medical issue and/or chief complaint. The resources include the type of medical vehicle, the medical personnel staffing the vehicle, vehicle equipment, such as patient lift assist, medical equipment, and medicine. The medical transfer protocol also objectively calculates a determinant code and a billing code at the time of the medical transfer request. Accordingly, uniform and consistent results are achieved through the disclosed system and variance due to human subjectivity is minimized.

While specific embodiments and applications of the disclosure have been illustrated and described, it is to be understood that the disclosure is not limited to the precise configuration and components disclosed herein. Various modifications, changes, and variations apparent to those of skill in the art may be made in the arrangement, operation, and details of the methods and systems of the disclosure without departing from the spirit and scope of the disclosure.

What is claimed is:

1. A computer system to perform a method to assist a dispatcher in responding to a caller requesting a medical transfer of a patient, comprising:
a processor;
an input device in electrical communication with the processor;
an output device in electrical communication with the processor; and
a memory in electrical communication with the processor, and having stored thereon:
a medical transfer protocol comprising:
a logic tree to present on the output device a pre-scripted interrogation comprising a plurality of preprogrammed inquiries for the dispatcher to ask the caller to systematically obtain a description of the medical transfer, wherein the description of the medical transfer comprises caller responses to the plurality of preprogrammed inquiries, the preprogrammed inquiries including asking whether a request is urgent or if the request is to schedule a medical transfer on a future day;
a determinant calculator to generate a determinant code to prioritize the medical transfer based on the caller responses; and
a billing code calculator to generate a billing code based on the caller responses and corresponding to the medical transfer.

2. The computer system of claim 1, wherein the preprogrammed inquires include asking if medication administration to the patient during the medical transfer is required.

3. The computer system of claim 1, wherein the preprogrammed inquires include asking if medical equipment for the patient during the medical transfer is required.

4. The computer system of claim 3, wherein the medical transfer protocol selects a medical vehicle based on the response to the medical equipment inquiry.

5. The computer system of claim 1, wherein the preprogrammed inquiries include asking the weight of the patient.

6. The computer system of claim 1, wherein the preprogrammed inquiries include asking if the patient is conscious and breathing.

7. The computer system of claim 1, wherein the preprogrammed inquires include asking the chief medical complaint of the patient.

8. The computer system of claim 1, wherein the medical transfer protocol provides post-dispatch instructions for the dispatcher to read to the caller.

9. The computer system of claim 1, further comprising a reporting module to measure the performance of the dispatcher.

10. A computer system to perform a method to assist a dispatcher in responding to a caller requesting a medical transfer of a patient, comprising:
a processor;
an input device in electrical communication with the processor;
an output device in electrical communication with the processor; and
a memory in electrical communication with the processor, and having stored thereon:
a medical transfer protocol comprising:
a logic tree to present on the output device a pre-scripted interrogation comprising a plurality of preprogrammed inquiries for the dispatcher to ask the caller to systematically obtain a description of the medical transfer, wherein the description of the medical transfer comprises caller responses to the plurality of preprogrammed inquiries, the preprogrammed inquiries including,
whether the request for medical transfer is urgent,
whether the request is to schedule a medical transfer on a future day, and
whether the request is for a mental health transfer;
a determinant calculator to generate a determinant code to prioritize the medical transfer based on the caller responses; and
a billing code calculator to generate a billing code based on the caller responses and corresponding to the medical transfer.

11. The computer system of claim 10, wherein the preprogrammed inquires include asking if medication administration to the patient during the medical transfer is required.

12. The computer system of claim 10, wherein the preprogrammed inquires include asking if medical equipment for the patient during the medical transfer is required.

13. The computer system of claim 12, wherein the medical transfer protocol selects a medical vehicle based on the response to the medical equipment inquiry.

14. The computer system of claim 10, wherein the preprogrammed inquiries include asking the weight of the patient.

15. The computer system of claim 10, wherein the preprogrammed inquiries include asking if the patient is conscious and breathing.

16. The computer system of claim 10, wherein the preprogrammed inquires include asking the chief medical complaint of the patient.

17. The computer system of claim 10, wherein the medical transfer protocol provides post-dispatch instructions for the dispatcher to read to the caller.

18. The computer system of claim 10, further comprising a reporting module to measure the performance of the dispatcher.

19. A computer readable medium comprising computer readable instruction code to perform a method for a medical transfer protocol to assist a dispatcher in responding to a caller requesting a medical transfer of a patient, the method comprising:
providing a logic tree to present on an output device a pre-scripted interrogation comprising a plurality of preprogrammed inquiries for the dispatcher to ask the caller to systematically obtain a description of the medical transfer, wherein the description of the medical transfer comprises caller responses to the plurality of preprogrammed inquiries, the preprogrammed inquiries including asking whether a request is urgent or if the request is to schedule a medical transfer on a future day;
generating a determinant code to prioritize the medical transfer based on the caller responses; and
generating a billing code based on the caller responses and corresponding to the medical transfer.

20. The computer readable medium of claim 19, wherein the preprogrammed inquires include asking if medication administration to the patient during the medical transfer is required.

21. The computer readable medium of claim 19, wherein the preprogrammed inquires include asking if medical equipment for the patient during the medical transfer is required.

22. The computer readable medium of claim 21, wherein the method further includes selecting a medical vehicle based on the response to the medical equipment inquiry.

23. The computer readable medium of claim 19, wherein the preprogrammed inquiries include asking the weight of the patient.

24. The computer readable medium of claim 19, wherein the preprogrammed inquiries include asking if the patient is conscious and breathing.

25. The computer readable medium of claim 19, wherein the preprogrammed inquires include asking the chief medical complaint of the patient.

26. The computer readable medium of claim 19, wherein the method further comprises providing post-dispatch instructions for the dispatcher to read to the caller.

27. The computer readable medium of claim 19, wherein the method further comprises measuring the performance of the dispatcher.
